(19) United States
(12) Patent Application Publication
(10) Pub. No.: US 2020/0173945 A1
(43) Pub. Date: Jun. 4, 2020

(54) CALIBRATION METHOD, THE USE THEREOF, AND APPARATUS FOR CARRYING OUT THE METHOD

(71) Applicant: Fraunhofer-Gesellschaft Zur Förderung Der Angewandten Forschung E.V., München (DE)

(72) Inventors: Andrea Burdack-Freitag, Holzkirchen (DE); Klaus Sedlbauer, Stuttgart (DE); Christian Scherer, Bad Aibling (DE); Maximilian Taubenberger, Warngau (DE); Leonhard Hillmeier, Valley (DE)

(21) Appl. No.: 16/612,143

(22) PCT Filed: May 3, 2018
(86) PCT No.: PCT/EP2018/061311

§ 371 Date: Nov. 8, 2019

(30) Foreign Application Priority Data
May 8, 2017 (DE) 10 2017 207 710.7
Jul. 10, 2017 (DE) 10 2017 211 731.1

(51) Int. Cl. G01N 27/02; G01N 27/12; G01N 33/00

(52) U.S. Cl. CPC G01N 27/026; G01N 27/126; G01N 33/0042; G01N 33/004; G01N 33/0006

(57) ABSTRACT

The application describes a method for calibrating metal oxide gas sensors using impedance spectroscopy, comprising the steps of: determining the impedance spectrum of the metal oxide gas sensor in a gas mixture in the absence of an analyte in order to determine a base line, and determining the impedance spectrum of the metal oxide gas sensor in the gas mixture in the presence of the analyte in at least a known concentration. The use of this method and an apparatus which can be used to carry out this method are also described.

### BACKGROUND

The invention relates to a method for calibrating metal oxide gas sensors, to the practical application of the method, and to an apparatus for carrying out the calibration method.

Metal oxide gas sensors (also referred to below as MOX sensors) are capacitive sensors. They consist of a reactive ceramic layer on a current-traversed carrier and depending on the metal oxide involved are capable of specific detection of gaseous substances (being known as metal oxide semiconductors). The principle of detection is based on changes in the surface (sensitive layer) and in the interior of the ceramic. A positive reaction of the gaseous substance with the MOX gas sensor is accompanied by a temperature-dependent change in the conductivity within the flow-traversed ceramic, which can be measured by a change in resistance.

The specificity for a particular gaseous substance is dependent on the chemical and physical nature of the MOX sensors (type of metal oxide, crystal structure, layer thickness, additional coating with sensitive layers, additional doping), the electronic parameters (input voltage, additional heating voltage) and the ambient conditions (background, baseline, oxygen content for regeneration, deactivating substances, ambient temperature, air pressure, humidity).

The conventional measurement of the specific conductivity is determined by applying a direct-current voltage, and is accompanied by the disadvantages of signal drift and of the long recovery times due to saturation of the surface. An absolute measurement of the concentration is not possible, because the baseline (the resistance measured without the analyte for measurement being present) is not reproducible. Metal oxide sensors used in the measurement of gaseous substances cannot be calibrated by the application of direct-current voltage. In the art this problem is countered by means of complicated arithmetic algorithms which calculate back to a baseline defined in the preceding period. In this way, relative changes in resistance are determined, allowing conclusions to be drawn about the change in concentration, depending on signal depth.

103 19 193 A1 discloses an apparatus and a method for determining concentrations of components of a gas mixture, having a sensitive layer which is disposed on a substrate and which varies its resistance depending on a concentration of a component of the gas mixture. The sensitive layer is configured such that a selectivity of the sensitive layer in relation to the components of the gas mixture can be altered in dependence on a frequency of an applied alternating-current voltage. This prior art, however, does not describe the use of an impedance curve as calibrating curve.

### SUMMARY OF THE INVENTION

Starting from the prior art, therefore, the object on which the invention is based is that of providing a method by which metal oxide gas sensors can be calibrated, where a reproducible baseline can be obtained, hence allowing absolute measurement of the concentration of the gas that is to be determined.

The object is achieved in accordance with the invention by a method as claimed in claim 1, by the use of the method as claimed in claim 15, by an apparatus as claimed in claim 16 and by a kit as claimed in claim 20. Advantageous developments of the invention are specified in the dependent claims.

Provided in accordance with the invention is a method for calibrating metal oxide gas sensors with impedance spectroscopy, comprising the steps of:

determining the impedance spectrum of the metal oxide gas sensor in a gas mixture in the absence of an analyte, to ascertain a baseline, and

determining the impedance spectrum of the metal oxide gas sensor in the gas mixture in the presence of the analyte in at least one known concentration.

The expression “calibrating” in the sense of the present invention means that a correlation between the output values and the known values is determined under defined conditions. This is done in the method of the invention by using impedance spectroscopy. Impedance spectroscopy determines the alternating-current resistance, also called impedance, as a function of the frequency of the alternating current. This is done by determining the impedance at a plurality of frequencies over a frequency range (spectrum). In one step the impedance spectrum of a gas mixture is determined without the analyte, i.e., without the substance under determination. Further, the impedance spectrum is determined in the presence of the analyte in the gas mixture, at a known concentration of the analyte. By plotting the measured impedance in an xy coordinate system against the frequency, the dependence of the measured impedance on the analyte concentration is obtained. Depending on concentration, for example, the impedance may be reduced in the presence of the analyte. If, subsequently, the MOX sensor calibrated in the steps described above is used for determining the analyte in unknown concentration in the gas mixture, it is then possible, by comparing the impedance determined in this case with the previously determined dependence of the impedance on the known concentrations, to determine the unknown concentration of the analyte.

As mentioned above, different frequency ranges are traversed by using impedance spectroscopy. The application of alternating current has a positive effect on signal stability. There is no saturation of the surface. The MOX sensor is immediately regenerated again. The baseline (i.e., the measurement without analyte) remains stable and there is no drift. By this means it is possible to calibrate the MOX sensors.

The measurements are reproducible. In definable concentration ranges and frequency intervals, the frequency-dependent component of the resistance is able to change in direct proportion to the concentration of substance in the gas mixture—in other words, calibration curves can be determined. It has been found that there are substance-specific frequencies which allow the determination of a limiting frequency, in order to make the sensors even more specific and to tailor them to specific substances. This allows the interconnection of a plurality of differently frequency-controlled sensors into a structure which is able to measure different substances and their concentration—so-called electronic noses.

On the basis of the model calibrated by impedance spectroscopy, it is possible for a microcontroller to be programmed for the control of metal oxide gas sensors and for a defined frequency to be entered individually in the microcontroller. The alternating-current voltage in this case need not be applied continuously; instead, it is possible to determine not only the frequency input but also the time intervals for the measuring points (e.g., one measurement value every 10 seconds). As a result, resistance values are obtained which indicate varying gas concentrations.

In certain embodiments the gas mixture used is selected from synthetic air and/or synthetic biogas and/or room air and/or inert gas and/or N₂ and/or at least one noble gas and/or N₂/CO and/or N₂/NOₓ and/or N₂/CO₂. When calibration takes place using one of these gas mixtures, important practical fields of application can be covered.

In certain embodiments the analyte is selected from water, carbon monoxide, alcohols, such as methanol, ethanol, 2-ethyl-1-hexanol and decanol, aldehydes, such as formaldehyde and acetaldehyde, ketones, such as acetone, 2-butanone, hexanal, octanal, decanal, acrolein, (E)-2-decenal, 6-methyl-5-hepten-4-one and 1-octen-3-one, terpenes, such as β-pinene, limonene and eucalyptol, organic acids, such as acetic acid and octanoic acid, aliphatic hydrocarbons, such as 1,3-pentadiene, isoprene, octane, alkane mixtures with 15 to 18 carbon atoms, thiols/sulfides, such as ethanediol, propanethiol, butanethiol, dimethyl sulfide, dimethyl trisulfide, methional and methylfurfurylthiol, esters, such as butyl acetate, compounds having an aromatic C6 group, such as benzene, toluene, p-cresol, 2-aminoacetophenone, acetonitrile and guaiacol, lactones, such as butyrolactone and octalactone, and halogenated organic compounds, such as dichloromethane. If the analyte is not in the form of a gas, it may be converted into a gas, by being vaporized, for example, before it is contacted with the MOX sensor.

In certain embodiments of the invention, the metal oxide gas sensor is selected from oxide ceramics, nonoxide ceramics, such as metal carbides, borides, silicides and nitrides, and clay minerals, such as zeolites and aluminosilicates. Examples of the oxide ceramic are SnO₂, AgO, CuO, Al₂O₃, WO₃, GeO₂, SiO₂, TiO₂, ZnO, In₂O₃ and Mn₂O₃ ceramics and mixtures of two or more of these, which may be doped with a metal selected from Pd, Pt, Au, Ag, Cd, Ni, Mn, Fe and Cu, for example, in an amount of about 0.2% to about 5%, based on the oxide ceramic. Specific examples are indicated in the table below:

| Ceramic | SnO₂ | AgO | CuO |
|---|---|---|---|
| No doping | X | X | X |
| Pd 0.2% | X | | |
| Pd 3% | X | X | X |
| Pt 0.2% | X | X | X |
| Ag 1% | X | | |
| Ag 3% | X | | |
| Cu 2% | X | | |
| Cu 5% | X | | |

X denotes that the metal oxide gas sensors are present in the specified concentrations

The metal oxide ceramics can be produced by calcination of the metal chlorides to give the corresponding metal oxides. Doping may be accomplished by adding metal chlorides corresponding to the doping metals in the requisite quantities prior to the heat treatment.

In certain embodiments the metal oxide gas sensor may have a coating, for example, with at least one compound selected from polymers, bioorganic substances, antibodies, metal-organic clusters, metal-organic framework compounds, metal organyls, ionic liquids, siloxanes and organic ions.

In certain embodiments at least one of the following impedance spectroscopy parameters may be selected: the impedance spectrum may be determined in a frequency range from about 1 to about 1 000 000 Hz, or about 100 to about 10 000. In the frequency range from about 100 to about 10 000 Hz, favorably, the resistance profile is at least approximately linear. The amplitude may be about 1 mV to about 12 V, or about 100 mV. Higher amplitudes, of about 500 mV, for example, may lead to a fluctuating resistance profile.

The impedance spectrum may be determined under at least one of the following conditions: the relative humidity may be about 0.7% to about 100%, or about 15% to about 60% or about 40%-about 60%. The temperature may be from about −55° C. to about +85° C., or about 18° C.-about 25° C. The pressure may be established at about 570 hPa to about 1600 hPa, or about 940-about 1200 hPa.

As indicated above, in certain embodiments the impedance spectrum may be determined, for example, at a relative humidity of about 15% to about 60%. At this humidity it has been found that a better measurement can be carried out with the MOX sensors.

In the method of the invention, the impedance spectrum is determined in the presence of a known concentration of the analyte. In this case a number of concentrations different from one another may be used—for example, three different analyte concentrations. In certain embodiments the impedance spectrum may be determined at an analyte concentration of about 1 ppb to about 100 ppb, or about 1 ppb, about 10 ppb and about 100 ppb. Surprisingly it has been found that calibration is possible in these low analyte concentrations. In this way it is possible to calibrate the MOX sensors for low concentrations and then to use them for measurements of the analytes at such low concentrations.

The above-described calibration method can be used for calibrating MOt sensors for a host of applications, as for example for regulating air supply in line with demand, for VOC measurement, in thermal processes, such as flue gas desulfurization, oxygen demand in combustion processes, for measuring ammonia and sulfur gas (determining the concentration of thiols and sulfides), for controlling technical fermentation processes, e.g., biogas production and bioethanol production, in food production, such as cheese ripening and yoghurt production, for example, in gas warning systems, in the case of carbon monoxide alarms, hydrogen sulfide alarms and nitrogen oxide alarms, for example, in military and security technology, for identifying hazardous substances, for example, in the realm of private and public transport, and for demand warning in vehicles—warning, for example, that a child has been forgotten in the vehicle, or as an alcohol barrier.

A further subject of the invention is an apparatus for calibrating metal oxide gas sensors with impedance spectroscopy, as for example by the method described above, comprising:

a measuring chamber with a metal oxide gas sensor,

means for determining the impedance spectrum, and

a metering facility for metering the gas mixture and optionally the analyte into the measuring chamber, the metering facility being connected via a line to the measuring chamber.

In certain embodiments the metering facility may comprise a first metering apparatus for the gas mixture and a second metering apparatus for the analyte.

In certain embodiments the second metering apparatus may be adapted to vaporize the analyte to allow it to be introduced as gas into the measuring chamber.

In certain embodiments the apparatus may further comprise a humidifying facility which is connected to the measuring chamber via a line, in order to establish a prespecified humidity in the measuring chamber.

As observed above, the method of the invention can be used to obtain and also record a calibration curve, i.e., it may be fixed on a carrier. By plotting the measured impedance in an xy coordinate system against the frequency, a dependency of the measured impedance on the concentration of the analyte is obtained. Depending on concentration, for example, the impedance may be reduced in the presence of the analyte. If, subsequently, the MOX sensor calibrated in the steps described above is used for determining the analyte in unknown concentration in the gas mixture, then it is possible, by comparing the impedance determined in this case with the previously determined dependence of the impedance on the known concentrations, to determine the unknown concentration of the analyte. If, therefore, the calibration curve of a metal oxide gas sensor is present, it can be used in order to permit quantitative measurements. Accordingly, the present invention provides a kit which comprises the recorded calibration curve, as obtained by the method of the invention, together with a metal oxide gas sensor calibrated therewith. For this purpose, the recorded calibration curve may be recorded on paper or on an electronic data medium.

### BRIEF DESCRIPTION OF THE DRAWINGS

The intention below is to elucidate the invention in more detail using figures and exemplary embodiments, without restricting the general concept of the invention. Here

FIG. 1 shows an apparatus of the invention.

FIG. 2 shows a metal oxide gas sensor as may be used in the apparatus of FIG. 1.

FIGS. 3a-3c show the calibration of a Pd/SnO₂ metal oxide gas sensor with a thiol and synthetic air.

FIGS. 4a-4c show the calibration of a Pd/SnO₂ metal oxide gas sensor with a sulfide and synthetic air.

FIGS. 5a-5c show the calibration of a CuO metal oxide gas sensor with a thiol and synthetic air.

FIGS. 6a-6c show the calibration of a CuO metal oxide gas sensor with a sulfide and synthetic air.

FIGS. 7a and 7b show the calibration of a Pd/SnO₂ metal oxide gas sensor with a thiol and synthetic biogas.

FIGS. 8a and 8b show the calibration of a sulfide with a Pd/SnO₂ metal oxide gas sensor and synthetic biogas.

FIGS. 9a-9j show the cross-correlation of a calibrated Pd/SnO₂ metal oxide sensor with various substances in synthetic air.

### DETAILED DESCRIPTION

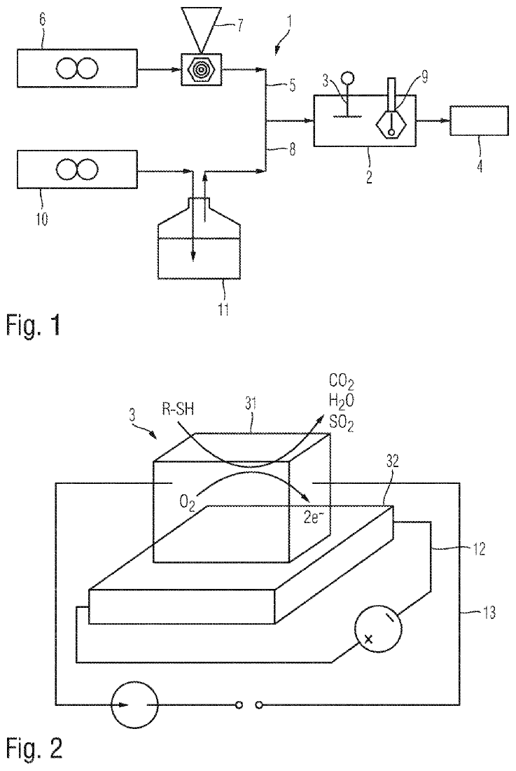

FIG. 1 serves to elucidate an apparatus of the invention with which the method of the invention can be carried out. The apparatus comprises a measuring chamber 2, in which the metal oxide gas sensor 3 is located. Further, the measuring chamber may contain a temperature/humidity/pressure sensor 9. Using this temperature/humidity/pressure sensor 9, it is possible to control the chamber conditions, which may be adjusted, for example, to about 0.7% to about 100%, or about 15% to about 60%, or about 40-about 60% relative humidity, a temperature of about −55° C. to about +85° C., about 18° C.-about 25° C. and a pressure of about 570 hPa to about 1600 hPa, or about 940-about 1200 hPa, to allow these chamber conditions to be reestablished in the event of deviations. For the sensors in the measuring chamber 2, steel or glass tube T-pieces may be used as sensor passages. The measuring chamber 2 may have an inert surface, achievable for example by powder coating, anodizing or siloxing. Measuring chambers 2 used may further comprise flow tubes and glass vessels, of the kind employed as laboratory reactors. The volume of the measuring chamber may be about 10 cm³ to about 2000 m³, as are used in the case of emissions testing chambers, for example. The measuring chamber may be connected to a detector 4, examples being FID (flame ionization detector), PID (photoionization detector), GC-MS (gas chromatography coupled with mass spectrometry), PTR-MS (photon transfer reaction mass spectrometry), FT-IR (Fourier-transform infrared spectrometer) and online NMR (nuclear magnetic resonance spectroscopy), in order to allow further measurements to be performed in order to determine the analyte.

The apparatus according to FIG. 1 further comprises a metering facility for metering the gas mixture and the optional analyte into the measuring chamber 2, the metering facility being connected via a line 5 to the measuring chamber 2. In FIG. 1, the first metering apparatus 6 of this metering facility comprises a mass flow regulator, allowing regulated supply of the background gas—for example, synthetic air or synthetic biogas. The flow rate may be, for example, about 1 ml/minute to about 100 L/minute, or about 2-about 3 L/min. This mass flow regulator may be connected via a line to a metering unit as second metering apparatus 7 for the analyte, said unit being connected via a line 5 to the measuring chamber 2. The second metering unit 7 may allow the metering of liquid by microdrop, or a piezoelectric crystal may be used as vaporizer.

The apparatus of FIG. 1 additionally has a humidifying apparatus, which in the present case comprises a mass flow regulator 10 whose mass flow is passed through a wash bottle 11 which is connected via a line 8 to the measuring chamber. The mass flow for the mass flow regulator 6 of the humidifying apparatus may be 0.4-0.5 L/min.

FIG. 2 shows a metal oxide gas sensor 3. In this case there is a ceramic layer 31, examples being the ceramics set out above, located on a heated carrier 32. A metal oxide gas sensor of this kind allows the impedance spectrum to be determined, for example, in a range from about 100 Hz to about 1 000 000 Hz. In FIG. 2, the reaction equation is shown schematically, for better illustration of the reaction occurring in the metal oxide gas sensor. Furthermore, FIG. 2 also shows the circuit 12 for the heating of the carrier 32 and the circuit 13 for the impedance measurement.

In the examples below, the impedance spectrum of various organic compounds was determined.

### Example 1

The substances were investigated in the apparatuses described in FIGS. 1 and 2 above. The conditions in the measuring chamber were 21° C./50% relative humidity/940 hPa. The background, i.e., the gas mixture used for determining the baseline (without analyte), and into which the analytes were then metered in concentrations of 1 ppb, 10 ppb and 100 ppb, were either synthetic air (20% O₂ and 80% N₂) or synthetic biogas with 60% methane, 38% CO₂ and 2% O₂. The overall volume flow was 2.51/min.

In preliminary investigations with direct-current measurement, the two sensor types SnO₂ with 3% Pd and pure CuO were found to be the most sensitive for sulfur-organic compounds. They were operated with a heating voltage of 2.7 V and over a frequency range of 100 to 1 000 000 Hz (amplitude 100 mV).

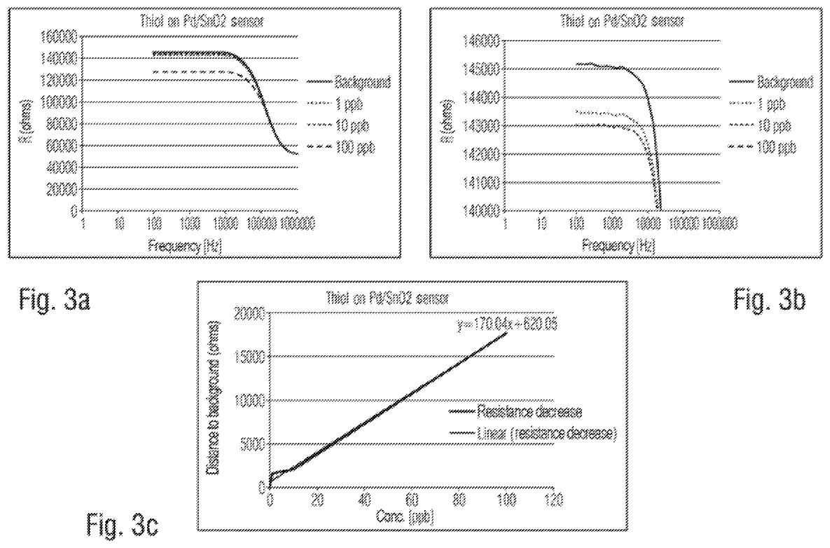

FIGS. 3a-3c show the calibration of the metal oxide gas sensor composed of SnO₂ with 3% Pd (referred to hereinafter as Pd/SnO₂ sensor) with butanethiol, the background used for determining the baseline being synthetic air. In FIG. 3a, the resistance R in ohms is plotted over the frequency range investigated. Here, in a first measurement, the baseline was determined, i.e., the impedance spectrum of the background, i.e., of the synthetic air, without the addition of butanethiol as analyte. This is described as “Background” in the figures. Then the impedance spectra at 1 ppb, 10 ppb and 100 ppb of butanethiol as analyte were determined. The impedance spectra, as frequency [Hz] versus R [ohms], are represented in FIG. 3a. For better illustration for the spectra found at low concentrations, the relevant part from FIG. 3a has been shown in enlarged form in FIG. 3b. In FIG. 3c, the concentration is plotted over the distance to the baseline (“Background”) in ohms. From FIG. 3c, it is possible to recognize a virtually linear curve profile which can be used as a calibration curve. If the sensor calibrated in this way is used for determining an unknown concentration of butanethiol by impedance spectroscopy, then the concentration can be taken directly, on the basis of the impedance determined, from FIG. 3c.

### Example 2

Example 2 was carried out in a similar way to example 1, with the difference that the analyte used was dimethyl sulfide.

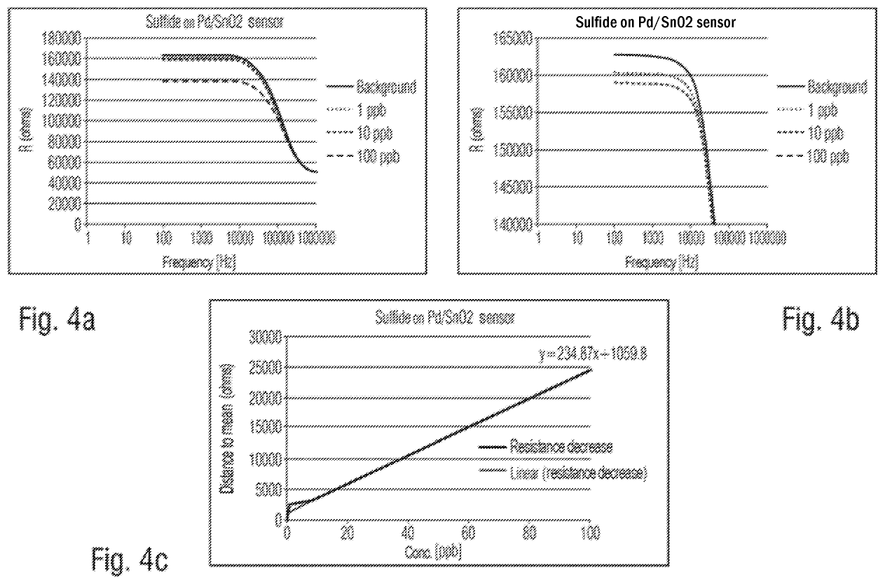

The results are shown in FIGS. 4a-4c. The results obtained were analogous to those in example 1, and accordingly reference is made to the comprehensive discussion of FIGS. 3a-3c above.

### Example 3

Example 3 was carried out in a similar way to example 1, with the difference that the metal oxide gas sensor used was CuO.

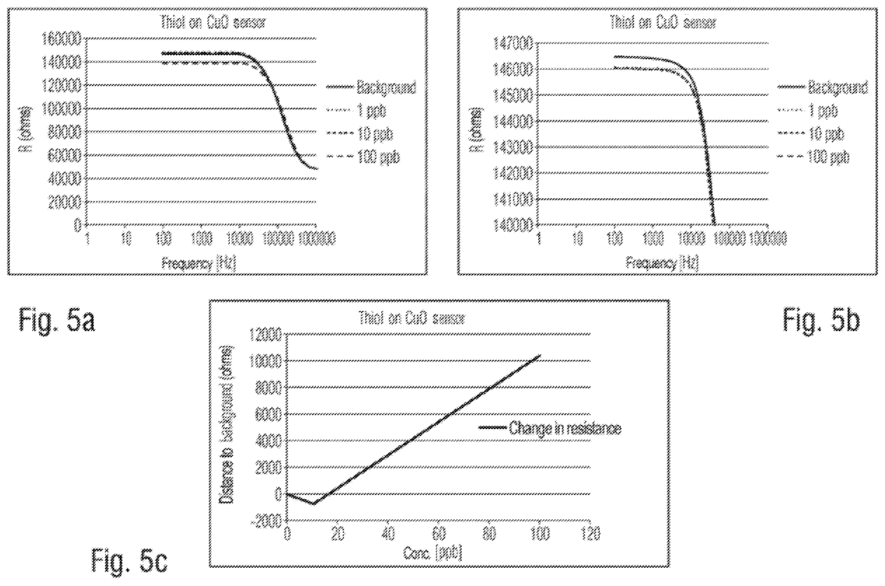

The results are shown in FIGS. 5a-5c. These correspond, in their implementation and in the result, to the above-discussed FIGS. 3a-3c, and hence for the interpretation of FIGS. 5a-5c, reference is made to the observations above.

### Example 4

Example 4 was carried out in a similar way to example 2, with the difference that the metal oxide sensor used was CuO.

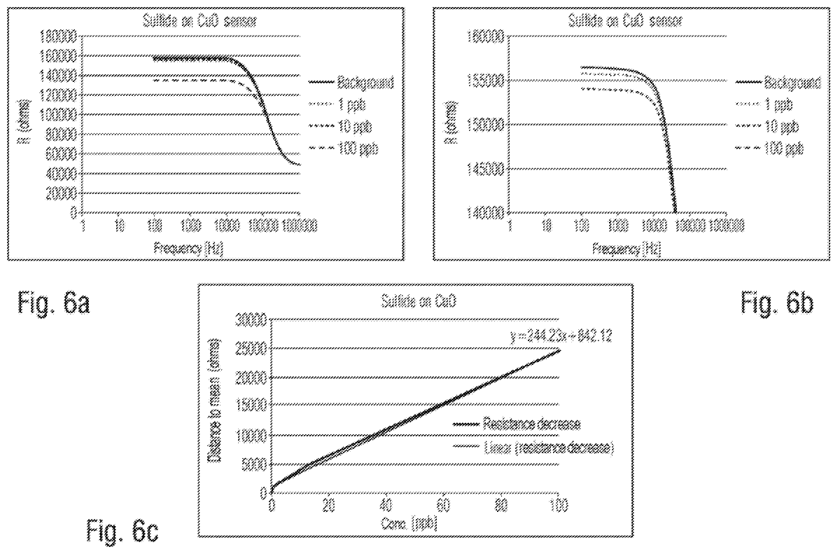

The results are shown in FIGS. 6a-6c. These correspond, in their implementation and in the result, to the above-discussed FIGS. 3a-3c, and hence for the interpretation of FIGS. 6a-6c, reference is made to the corresponding observations above.

### Example 5

Example 5 was carried out in a similar way to example 1, with the difference that synthetic biogas with 60% methane, 38% CO₂ and 2% O₂ was used instead of synthetic air.

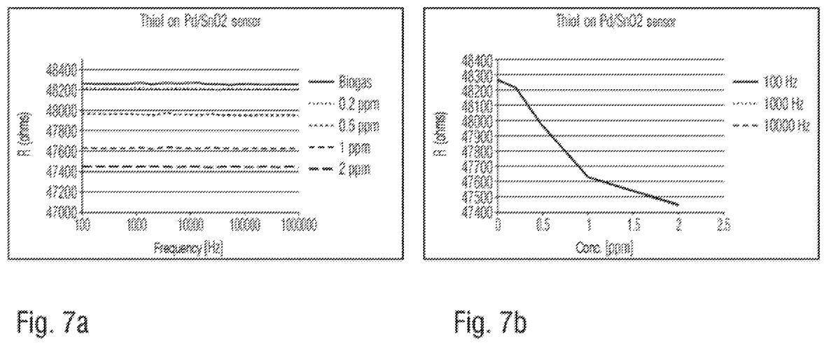

The results are shown in FIGS. 7a and 7b. Here, FIG. 7a shows the resistances found relative to the frequency range investigated, in the concentrations indicated in the figures, and FIG. 7b shows in graph form the concentration relative to the resistance, at the specified frequencies.

### Example 6

Example 6 was carried out in a similar way to example 2, with the synthetic biogas indicated in example 5 being used instead of the synthetic air.

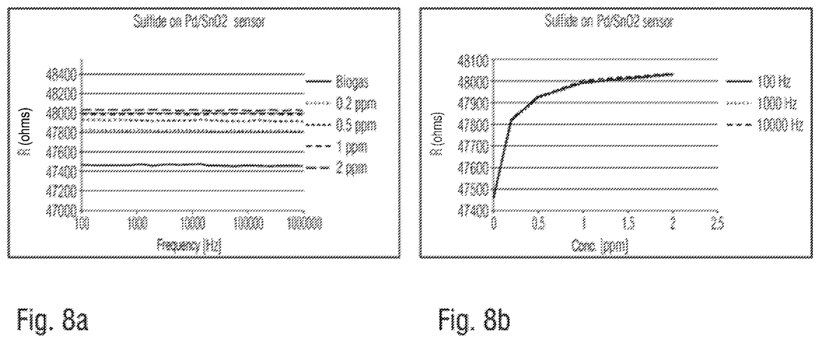

The results are shown in FIGS. 8a and 8b. Here, FIG. 8a shows the resistance found relative to the frequency applied, at the concentrations indicated in FIG. 8a. In FIG. 8b, these concentrations are plotted against the resistance found, at the specified frequencies.

### Example 7

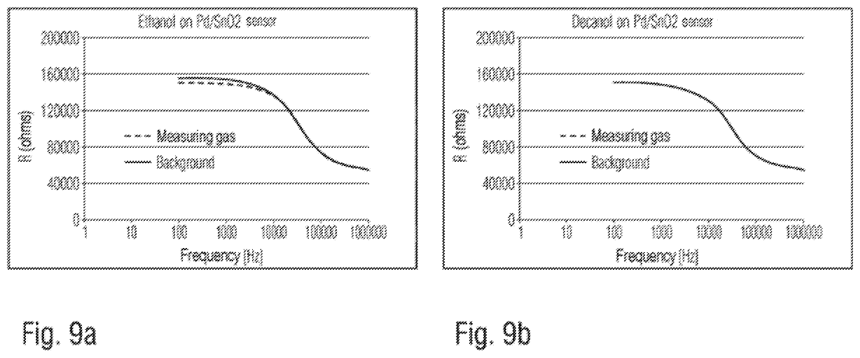

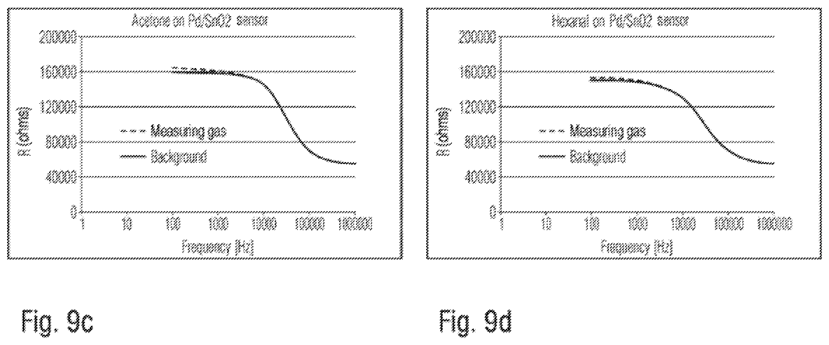

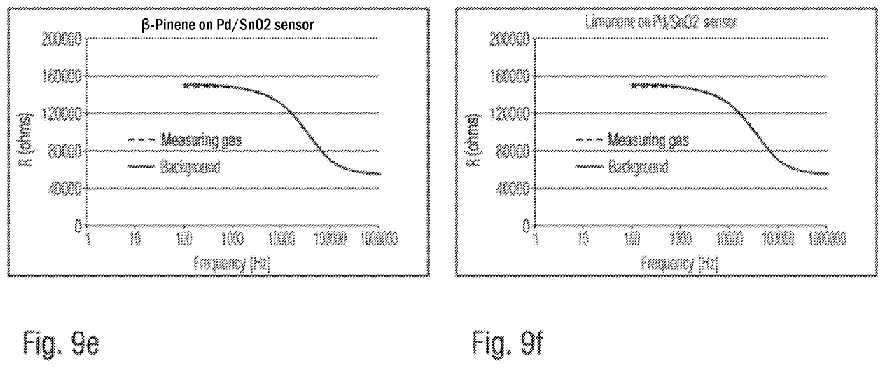

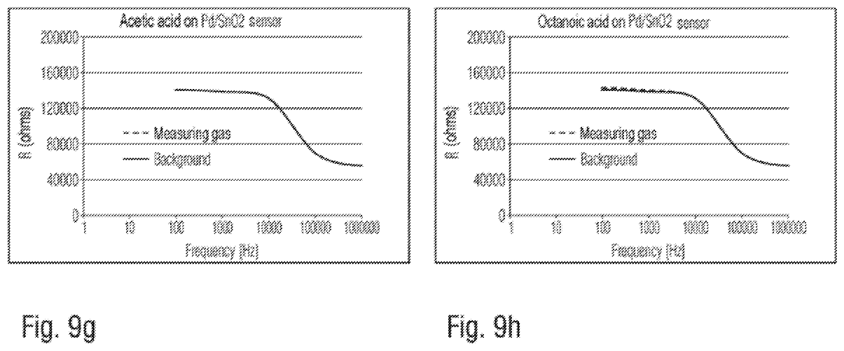

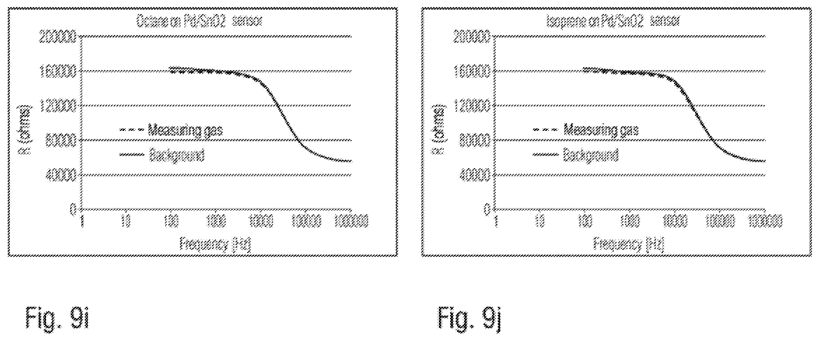

In example 7, the substances ethanol, decanol, acetone, hexanal, β-pinene, limonene, acetic acid and octanoic acid, octane and isoprene as analyte were investigated by impedance spectroscopy using SnO₂ with 3% Pd as metal oxide gas sensor against the background of synthetic air. The above analytes were metered in a concentration of 100 ppb. The results are shown in FIGS. 9a-9j.

The results show that with a specified sensor, it is possible to test other analytes, in order to determine the sensitivity of the sensor for the other analytes.

The invention is of course not confined to the examples and embodiments represented in the figures. The above description should therefore be regarded not as restricting, but instead as illustrative. The claims which follow should be understood to mean that a stated feature is present in at least one embodiment of the invention. This does not exclude the presence of further features. Where the claims and the above description define “first” and “second” features, this designation serves for distinguishing two features of the same kind, without specifying any hierarchy.

1. A method for calibrating metal oxide gas sensors with impedance spectroscopy, comprising the steps of:
determining the impedance spectrum of the metal oxide gas sensor in a gas mixture in the absence of an analyte, to specify a baseline, and
determining the impedance spectrum of the metal oxide gas sensor in the gas mixture in the presence of the analyte in at least one known concentration.

2. The method as claimed in claim 1, wherein the gas mixture is selected from synthetic air and/or synthetic biogas and/or room air and/or inert gas and/or N₂ and/or at least one noble gas and/or N₂/CO and/or N₂/NOₓ and/or N₂/CO₂.

3. The method as claimed in claim 1, wherein the analyte is selected from water and/or carbon monoxide and/or at least one alcohol and/or at least one aldehyde, and/or at least one ketone and/or at least one terpene and/or at least one organic acid and/or at least one aliphatic hydrocarbon and/or at least one thiol and/or at least one sulfide and/or at least one ester and/or at least one compound having an aromatic C6 group and/or at least one lactone and/or at least one halogenated organic compound.

4. The method as claimed in claim 1, wherein the metal oxide gas sensor is selected from oxide ceramics, nonoxide ceramics and clay minerals.

5. The method as claimed in claim 4, wherein the oxide ceramic is selected from at least one SnO₂, AgO, CuO, Al₂O₃, WO₃, GeO₂, SiO₂, TiO₂, ZnO, In₂O₃ or Mn₂O₃ ceramic or mixture of at least two of the stated compounds.

6. The method as claimed in claim 5, wherein the oxide ceramic is doped with at least one metal.

7. The method as claimed in claim 6, wherein the metal is selected from Pd, Pt, Au, Ag, Cd, Ni, Mn, Fe and/or Cu and/or wherein the metal is incorporated in an amount of about 0.2 to about 5 wt %, based on the oxide ceramic.

8. The method as claimed in claim 1, wherein the metal oxide gas sensor is coated with at least one compound selected from at least one polymer, one bioorganic substance, one antibody, one metal-organic cluster, one metal-organic framework compound, one metal organyl, one ionic liquid, one siloxane and/or organic ions.

9. The method as claimed in claim 1, wherein the impedance spectrum is determined in a frequency range from about 1 Hz to about 1 000 000 Hz.

10. The method as claimed in claim 1, wherein the impedance spectrum is determined in a frequency range from about 100 Hz to about 10 000 Hz.

11. The method as claimed in claim 1, wherein the impedance spectrum is determined at an amplitude of about 1 mV to about 12 V.

12. The method as claimed in claim 1, wherein the impedance spectrum is determined at a relative humidity of about 15% to about 60%.

13. The method as claimed in claim 1, wherein the impedance spectrum is determined at an analyte concentration of about 1 to about 100 ppb.

14. The method as claimed in claim 1, wherein the analyte is contacted as gas with the metal oxide gas sensor.

15. The use of the method as claimed in claim 1 for regulating air supply in line with demand, for VOC measurement, in thermal processes, for measuring ammonia and sulfur gas, for controlling technical fermentation processes, in food production, in gas warning systems, in military and security technology, in the realm of private and public transport and for demand warning in vehicles.

16. An apparatus (1) for calibrating metal oxide gas sensors with impedance spectroscopy, comprising:
a measuring chamber (2) with a metal oxide gas sensor (3),
a facility for determining the impedance spectrum, and
a metering facility for metering the gas mixture and optionally the analyte into the measuring chamber (2), the metering facility being connected via a line (5) to the measuring chamber (2).

17. The apparatus as claimed in claim 16, wherein the metering facility comprises a first metering apparatus (6) for the gas mixture and a second metering apparatus (7) for the analyte.

18. The apparatus (1) as claimed in claim 16, wherein the second metering apparatus (7) is adapted to vaporize the analyte so that it is introduced as a gas into the measuring chamber (2).

19. The apparatus (1) as claimed in claim 16, which further comprises a humidifying facility which is connected to the measuring chamber (2) via a line (10) so as to establish a prespecified humidity in the measuring chamber (2).

20. A kit comprising the recorded calibration curve of claim 1 and a metal oxide gas sensor calibrated therewith.
